(19) United States
(12) Patent Application Publication
(10) Pub. No.: US 2025/0281798 A1
(43) Pub. Date: Sep. 11, 2025

(54) INFORMATION PROCESSING DEVICE AND INFORMATION PROCESSING METHOD

(71) Applicant: SEIKO EPSON CORPORATION, Tokyo (JP)

(72) Inventors: Suzuyo OHARA, CHINO-SHI (JP); Yusuke TAKAHASHI, MATSUMOTO-SHI (JP)

(73) Assignee: SEIKO EPSON CORPORATION, Tokyo (JP)

(21) Appl. No.: 19/071,764

(22) Filed: Mar. 6, 2025

(30) Foreign Application Priority Data
Mar. 8, 2024 (JP) 2024-035527

(51) Int. Cl. A63B 24/00; A61B 5/0205; A63B 71/06; G16H 10/60; G16H 20/30

(52) U.S. Cl. CPC A63B 24/0084; A61B 5/0205; A63B 71/0622; G16H 10/60; G16H 20/30; A63B 2024/0068

(57) ABSTRACT

An information processing device includes an acquisition unit configured to acquire biometric information of a subject, a determination unit configured to determine a state of the subject from the biometric information, a generation unit configured to generate advice corresponding to the state of the subject, and an output unit configured to output the advice generated, in which the output unit changes an output timing of the advice in accordance with the biometric information.

The present application is based on, and claims priority from JP Application Serial Number 2024-035527, filed Mar. 8, 2024, the disclosure of which is hereby incorporated by reference herein in its entirety.

### BACKGROUND

### 1. Technical Field

The present disclosure relates to an information processing device, an information processing method and a recording medium.

### 2. Related Art

The information processing device disclosed in JP-A-2016-198352 determines that the player is not exercising on the basis of information about the body movement of the player and information about the moving state of the equipment, and reports advice when the player is not exercising (see JP-A-2016-198352).

However, in the known technique, the biometric information of the subject is not acquired, and therefore the output timing of advice cannot be changed in accordance with the biometric information. For example, the output timing of advice cannot be changed in accordance with the exercise state on the basis of the biometric information. As such, in the known technique, advice may possibly be output at a timing that does not match the biometric condition of the subject, for example.

### SUMMARY

An information processing device according to an aspect to solve the above-mentioned problems includes an acquisition unit configured to acquire biometric information of a subject, a determination unit configured to determine a state of the subject from the biometric information, a generation unit configured to generate advice corresponding to the state of the subject, and an output unit configured to output the advice generated, in which the output unit changes an output timing of the advice in accordance with the biometric information.

An information processing method according to an aspect to solve the above-mentioned problems includes acquiring, by an acquisition unit, biometric information of a subject, determining, by a determination unit, a state of the subject from the biometric information, generating, by a generation unit, advice corresponding to the state of the subject, and outputting, by an output unit, the advice generated in such a manner that an output timing of advice is changed in accordance with the biometric information.

A recording medium according to an aspect to solve the above-mentioned problems is configured to record a program, in which the program causes a computer to execute an acquisition function of acquiring biometric information of a subject, a determination function of determining a state of the subject from the biometric information, a generation function of generating advice corresponding to the state of the subject, and an output function of outputting the advice generated in such a manner that an output timing of advice is changed in accordance with the biometric information.

### BRIEF DESCRIPTION OF THE DRAWINGS

FIG. 1 is a diagram schematically illustrating an example of a configuration of an information processing device according to an embodiment.

FIG. 2 is a diagram illustrating an example of classification of biometric information according to the embodiment.

FIG. 3 is a first table illustrating an example of details of heart rate zone according to the embodiment.

FIG. 4 is a diagram illustrating an example of a procedure of processes performed by the information processing device according to the embodiment.

FIG. 5 is a second table illustrating an example of patterns of a relationship between timing states and contents of advice according to the embodiment.

FIG. 6 is a third table illustrating an example of a tendency of biometric information serving as an estimation source related to each pattern according to the embodiment.

FIG. 7 is a graph illustrating an example of a tendency of a first pattern according to the embodiment.

FIG. 8 is a graph illustrating an example of a tendency of a second pattern according to the embodiment.

FIG. 9 is a graph illustrating an example of a tendency of a third pattern A according to the embodiment.

FIG. 10 is a graph illustrating an example of a tendency of a third pattern B according to the embodiment.

FIG. 11 is a graph illustrating an example of a tendency of a fourth pattern according to the embodiment.

FIG. 12 is a graph illustrating an example of a tendency of a fifth pattern according to the embodiment.

FIG. 13 is a graph illustrating an example of a tendency of a sixth pattern according to the embodiment.

FIG. 14 is a graph illustrating an example of a tendency of a seventh pattern according to the embodiment.

FIG. 15 is a graph illustrating an example of a tendency of an eighth pattern A according to the embodiment.

FIG. 16 is a graph illustrating an example of a tendency of an eighth pattern B according to the embodiment.

### DESCRIPTION OF EMBODIMENTS

An embodiment is described below with reference to the accompanying drawings.

An embodiment is described below.

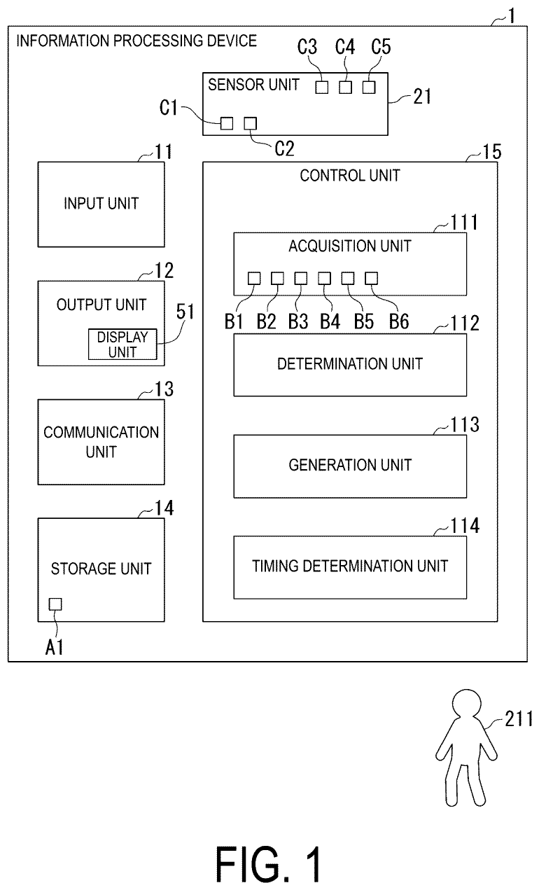

FIG. 1 is a diagram schematically illustrating an example of a configuration of an information processing device 1 according to the embodiment.

The information processing device 1 includes an input unit 11, an output unit 12, a communication unit 13, a storage unit 14, a control unit 15, and a sensor unit 21.

The output unit 12 includes a display unit 51.

The control unit 15 includes an acquisition unit 111, a determination unit 112, a generation unit 113, and a timing determination unit 114.

The acquisition unit 111 includes a biometric information acquisition unit B1, a body movement information acquisition unit B2, a behavior information acquisition unit B3, a location/movement information acquisition unit B4, a voice information acquisition unit B5, and a time information acquisition unit B6.

The sensor unit 21 includes a PPG (Photoplethysmography) sensor C1, a temperature sensor C2, a movement sensor C3, a GPS (Global Positioning System) sensor C4, and a sound sensor C5.

Note that other methods such as GNSS (Global Navigation Satellite System) may be used instead of GPS, for example.

In addition, FIG. 1 illustrates a subject 211 as a human.

In this embodiment, the subject 211 is a person who exercises.

In this embodiment, for example, the information processing device 1 is a wristwatch type device that is attached to an arm of the subject 211 when used, but the present disclosure is not limited to this. For example, the information processing device 1 may be a device that is attached to an upper arm, or a leg.

Note that the information processing device 1 is not limited, and may be smartphones, tablet terminals, or other computers, for example.

The input unit 11 performs a process of inputting information in accordance with the user operation, and a process of inputting information from other devices, for example.

The output unit 12 performs a process of outputting information to other devices, for example.

For example, the display unit 51 includes a screen for displaying and outputting information, and displays relevant information on the screen.

Note that the output unit 12 may output information to an external display device to display the information on a screen of the display device, or output information to an external printing device to print out the information from the printing device, for example.

In addition, the output unit 12 may output information in forms other than display such as voice and light by the information processing device 1 or through external devices, for example.

The communication unit 13 communicates with other devices.

Note that this embodiment describes the communication unit 13 separately from the input unit 11 and the output unit 12, but the function of the communication unit 13 may be included in the functions of the input unit 11 and the output unit 12, for example.

The storage unit 14 stores information.

In this embodiment, the storage unit 14 includes an advice content storage unit A1 that stores advice information including the content of the advice, for example.

Note that this embodiment describes a configuration in which the advice content storage unit A1 is included in the storage unit 14 for convenience of description, but the function of the advice content storage unit A1 may not necessarily be clearly sectioned in the storage unit 14.

In this embodiment, the storage unit 14 store that various information used in the processing of the information processing device 1, and may store that information detected by the sensor unit 21, information acquired by the acquisition unit 111, and other information, for example.

The control unit 15 performs various processes and controls.

In this embodiment, the information processing device 1 is configured to with a computer.

The control unit 15 includes a processor such as a CPU (Central Processing Unit), and performs various processes and controls by executing predetermined programs using the processor.

The programs may be stored in the storage unit 14, for example.

The acquisition unit 111 acquires various information.

In this embodiment, the acquisition unit 111 acquires at least one of biometric information, body movement information, behavior information, location/movement information, voice information, and time information.

For convenience of description, this embodiment describes a configuration in which the acquisition unit 111 includes the biometric information acquisition unit B1 that acquires biometric information that is biometric information about the subject 211, the body movement information acquisition unit B2 that acquires body movement information that is operation information about the subject 211, the behavior information acquisition unit B3 that acquires behavior information of the subject 211, the location/movement information acquisition unit B4 that acquires the location/movement information of the subject 211, the voice information acquisition unit B5 that acquires voice information, and the time information acquisition unit B6 that acquires time information, but these functions of the information acquisition units may not necessarily be clearly sectioned in the acquisition unit 111.

In addition, on the basis of one or more pieces of acquired information, the acquisition unit 111 may acquire still other pieces of information. The other pieces of information may be information such as the exercise intensity of the subject 211 and the exercise amount of the subject 211, for example. In this embodiment, the exercise intensity of the subject 211, the exercise amount of the subject 211, or a combination of them may be used as an index for determining the output timing of advice. Note that the output timing of advice may be determined using the index and other indexes, for example.

Note that the output timing of advice may be referred to as the presenting timing of advice, the report timing of advice, the report timing of advice and the like.

Here, the behavior information acquisition unit B3 may acquire information about daily behavior history of the subject 211, for example.

In addition, the location/movement information acquisition unit B4 may acquire the location information of the subject 211 and the movement history information of the subject 211.

In addition, the voice information acquisition unit B5 may acquire voice information around the subject 211, for example.

The determination unit 112 determines the state of the subject 211 on the basis of biometric information acquired by the acquisition unit 111.

The generation unit 113 generates advice corresponding to the state of the subject 211. In this embodiment, the advice information is stored and held in the advice content storage unit A1. Note that the advice information includes information about the content of the advice.

The timing determination unit 114 determines the output timing of advice on the basis of a predetermined determination index. In this embodiment, the determination index includes at least one of the biometric information of the subject 211 and other information obtained from the biometric information, for example.

Here, in this embodiment, the output unit 12 outputs advice at the timing determined by the timing determination unit 114.

In addition, in this embodiment, the output unit 12 outputs advice including the content of the advice stored in the advice content storage unit A1.

In this manner, the output unit 12 can change the output timing of advice in accordance with the biometric information of the subject 211.

The PPG sensor C1 detects pulse information, SpO2 information and the like of the subject 211.

The temperature sensor C2 detects body temperature information of the subject 211.

The movement sensor C3 detects movement information of the subject 211. In this embodiment, the movement information of the subject 211 is used as the behavior information of the subject 211.

The GPS sensor C4 detects the position information of the subject 211 by using the GPS.

The sound sensor C5 detects sound information. In this embodiment, the sound includes voice.

Here, the sensor unit 21 includes at least one sensor capable of acquiring the biometric information of the subject 211. In other points, the type, number or the like of sensors provided in the sensor unit 21 is not limited, and various configurations may be used.

In addition, this embodiment describes an exemplary configuration in which all sensors are included in the sensor unit 21 for convenience of description, but this is not limitative, and each sensor may be independently provided, for example.

Note that the sensor may be referred to as a detection unit or the like.

The biometric information acquisition unit B1 acquires biometric information on the basis of at least one of information about the detection result of the PPG sensor C1 and information about the detection result of the temperature sensor C2.

The body movement information acquisition unit B2 acquires body movement information on the basis of information about the detection result of the movement sensor C3.

The behavior information acquisition unit B3 acquires behavior information on the basis of at least one of information about the detection result of the movement sensor C3 and information about the detection result of the GPS sensor C4.

The location/movement information acquisition unit B4 acquires the location information or movement information on the basis of information about the detection result of the GPS sensor C4.

The voice information acquisition unit B5 acquires voice information on the basis of information about the detection result of the sound sensor C5.

The time information acquisition unit B6 acquires time information with information about the detection result of the GPS sensor C4 or a clock function provided in the information processing device 1, for example. Note that the time information acquisition unit B6 may acquire time information on the basis of time signal information provided by an external device, for example.

Here, the time information may include information about time of day and information about the length of time, for example.

As a specific example, through measurement, the PPG sensor C1 a detects the sensor information from which information such as pulse information, SpO2 information, sleep information, and respiration information is calculated.

As a specific example, through measurement, the temperature sensor C2 detects the sensor information from which the body temperature information is calculated.

As a specific example, the biometric information acquisition unit B1 of the acquisition unit 111 calculates pulse information, SpO2 information, sleep information, respiration information, body temperature information, and the like.

For example, the body movement information acquisition unit B2 and the timing determination unit 114 can determine the output of advice at a timing when the subject 211 is not moving. Note that when the subject 211 is not moving, it is estimated that the subject 211 is resting.

For example, the behavior information acquisition unit B3 and the timing determination unit 114 can store the time when the subject 211 normally exercises and the like, and can determine the output of advice at a timing slightly before the timing when the subject 211 exercises.

For example, the location/movement information acquisition unit B4 and the timing determination unit 114 can estimate the start of exercise from the location of the subject 211, and can determine the output of advice at the estimated timing.

For example, the voice information acquisition unit B5 and the timing determination unit 114 can determine the output of advice at a timing when the subject 211 is not conversing with others.

For example, the voice information acquisition unit B5 and the timing determination unit 114 can determine the output of advice at a timing when the subject 211 is making positive statements.

For example, the time information acquisition unit B6 and the timing determination unit 114 can determine the output of advice at a timing when a predetermined biometric state has continued for a certain period.

For example, the time information acquisition unit B6 and the timing determination unit 114 can determine the output of advice at a timing when a predetermined time has elapsed after the detection of a predetermined biometric state.

For example, the time information acquisition unit B6 and the timing determination unit 114 can measure the elapse of time for the report after falling asleep and after waking up, and can output advice at a timing when a predetermined time has elapsed.

Note that falling asleep may be referred to as bedtime.

This embodiment describes the information processing device 1 of an integrated type for convenience of description, but the functions of the information processing device 1 illustrated in FIG. 1 may be provided in a distributed manner in a plurality of devices, i.e., the information processing device 1 illustrated in FIG. 1 may be composed of two or more devices, for example. In the case where the information processing device 1 is composed of two or more devices, the two or more devices communicate with each other in a wired or wireless manner as necessary, for example.

As an example, a device including the function of the sensor unit 21 illustrated in FIG. 1 may be composed of a member separated from the information processing device 1.

In addition, this embodiment describes a case where the information processing device 1 is attached to an arm of the subject 211, but as another example, the information processing device 1 may have another configuration and attached to one or more places other than the arm of the subject 211.

In addition, the information processing device 1 may have an AI (Artificial Intelligence) function and use it.

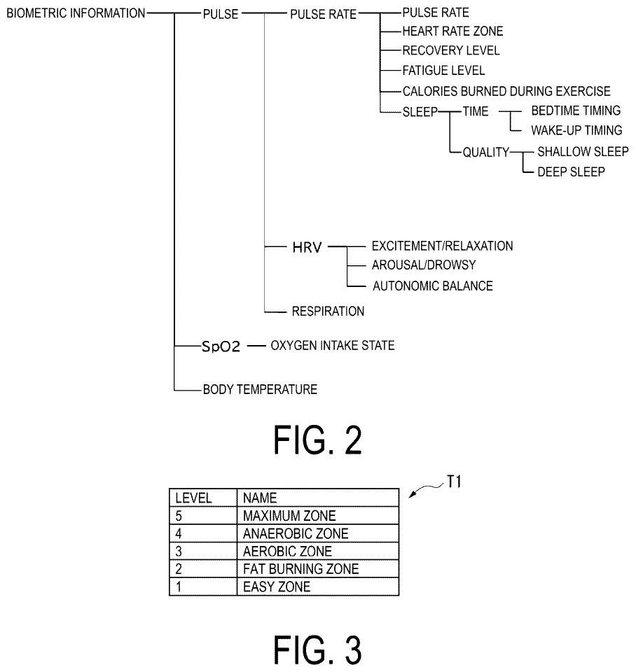

FIG. 2 is a diagram illustrating an example of classification of biometric information according to the embodiment.

In the example illustrated in FIG. 2, biometric information includes information such as pulse, SpO2, which is transcutaneous arterial blood oxygen saturation, and body temperature.

From the pulse information, information such as pulse rate, HRV (Heart Rate Variability), and respiration can be obtained.

From the pulse rate information, information such as pulse rate, heart rate zone, recovery level, fatigue level, calories burned during exercise, and sleep can be obtained.

From the sleep information, information such as sleep time, and sleep quality can be obtained.

From the sleep time information, information such as bedtime timing and wake-up timing can be obtained.

From the sleep quality information, information such as information indicating whether sleep is shallow sleep or deep sleep can be obtained.

From the HRV information, information such as excitement/relaxation, arousal/drowsiness, and autonomic balance can be obtained.

From the SpO2 information, information such as oxygen intake state can be obtained.

Note that the example illustrated in FIG. 2 is merely an exemplification for description, and the present disclosure is not limited to this. Various biometric information may be used, and various biometric information may be acquired in various ways.

FIG. 3 is a first table T1 illustrating an example of details of the heart rate zone according to the embodiment.

In the first table T1, the level and name are stored in association with one another as details of the heart rate zone.

The level 1 is an easy zone, the level 2 is a fat burning zone, the level 3 is an aerobic zone, the level 4 is an anaerobic zone, and the level 5 is a maximum zone.

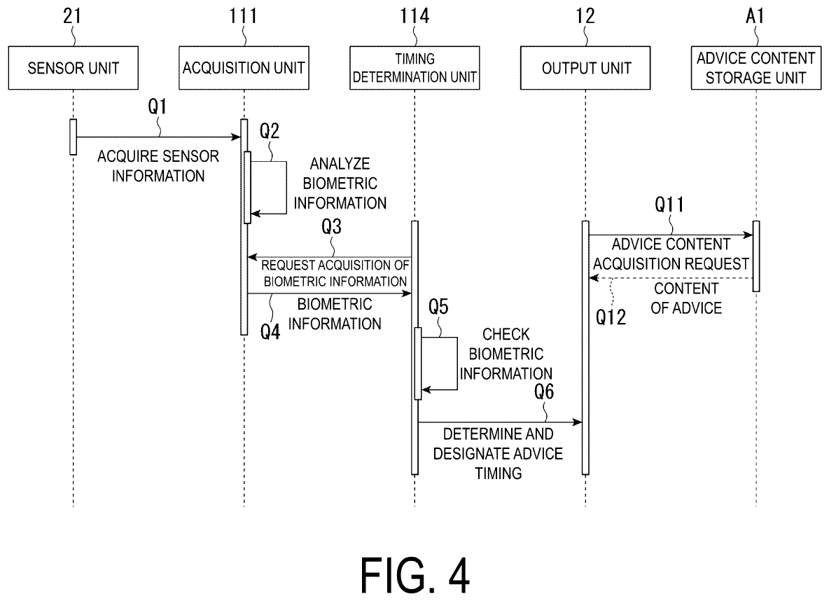

FIG. 4 is a diagram illustrating an example of a procedure of processes performed by the information processing device 1 according to the embodiment.

In the example illustrated in FIG. 4, outlines of the processes from acquisition of the sensor information to determination and designation of the advice timing are illustrated.

Note that the example illustrated in FIG. 4 is merely an example, and the present disclosure is not limited to this, and, various processing procedures may be used.

In a process Q1, the sensor unit 21 acquires sensor information and sends it to the sensor information the acquisition unit 111.

In a process Q2, the acquisition unit 111 analyzes biometric information based on the sensor information to acquire predetermined information. Here, the predetermined information is information that can be used for the report of advice in the various information illustrated in FIG. 2.

In a process Q3, the timing determination unit 114 sends a biometric information acquisition request to the acquisition unit 111. In a process 04, the acquisition unit 111 sends the requested biometric information to the timing determination unit 114 in response to the reception of the biometric information acquisition request.

In a process Q5, the timing determination unit 114 checks the biometric information received from the acquisition unit 111.

In a process Q6, the timing determination unit 114 determines the timing of advice on the basis of the check result of the biometric information, and sends the timing to the output unit 12 for designation.

In addition, processes Q11 and Q12 described below may be performed at timings independent of, or linked with, the above-described processes.

In the process Q11, the output unit 12 sends an advice content acquisition request to the advice content storage unit A1.

In the process Q12, in response to the advice content acquisition request, the output unit 12 acquires from the advice content storage unit A1 the content of the advice requested in the advice content acquisition request. In this manner, the output unit 12 outputs advice of the acquired content of the advice at a designated timing.

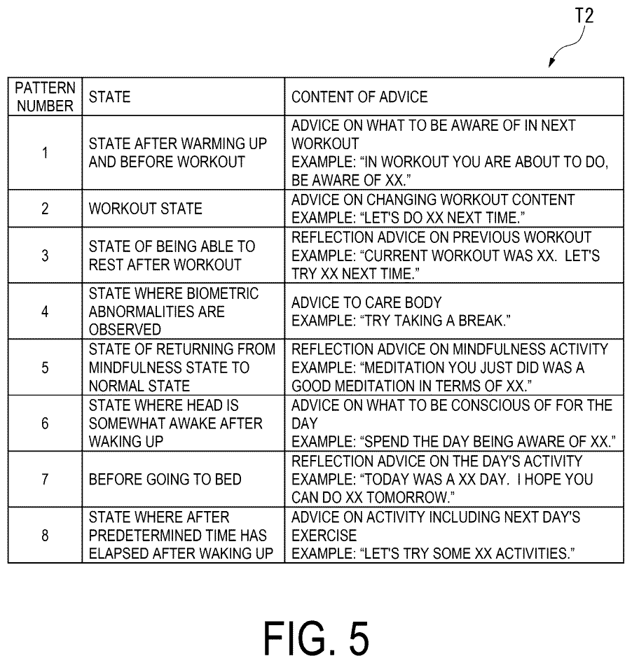

FIG. 5 is a second table T2 illustrating an example of patterns of a relationship between timing states and contents of advice according to the embodiment.

In this embodiment, examples of a first pattern to an eighth pattern are described.

The second table T2 stores the pattern number, the state of the subject 211, and the content of the advice to be output in association with one another.

Here, the state of the subject 211 is a state estimated from the biometric information of the subject 211, and in this example, a state determined as the output timing of advice.

In addition, the content of the advice includes information about the type of the advice, and specific exemplary sentences.

Note that the example illustrated in FIG. 5 is merely an example, and the present disclosure is not limited to this, and, various configurations may be used.

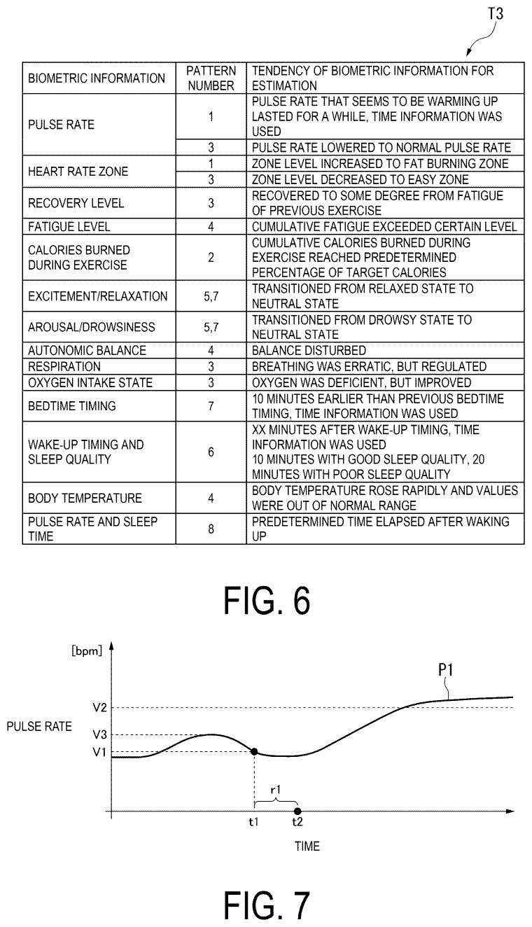

FIG. 6 is a third table T3 illustrating an example of a tendency of biometric information serving as an estimation source related to each pattern according to the embodiment.

In the third table T3, the biometric information of the subject 211, the pattern number, and the tendency of biometric information serving as the estimation source are stored in association with one another.

Note that the example illustrated in FIG. 6 is merely an example, and the present disclosure is not limited to this, and, various configurations may be used.

Here, for example, the state illustrated in the second table T2 may be estimated on the basis of the tendency of biometric information serving as the estimation source illustrated in the third table T3.

Note that if the same pattern number exists in two or more places in the third table T3, the tendency of any one of the places may be used, or the tendencies of any two or more of the places may be used in combination.

FIG. 7 is a graph illustrating an example of a tendency of a first pattern according to the embodiment.

In the graph, the abscissa indicates time, and the ordinate indicates a pulse rate value [bpm]. In the graph, a first characteristic P1 representing a relationship between time and pulse rate is illustrated. The first characteristic P1 represents tendency of transition of pulse rate with time.

In addition, in the graph, a first value V1, which is a pulse rate value, a second value V2, which is another pulse rate value, and a third value V3, which is still another pulse rate value, are illustrated. The second value V2 is greater than the first value V1 and the third value V3. The third value V3 is greater than the first value V1.

In this example, a state where the pulse rate is equal to or greater than first value V1 and is smaller than second value V2 is considered a warming-up state, and a state where the pulse rate is equal to or greater than second value V2 is considered a workout state.

In the first pattern, the output timing of advice is a timing after the warming-up and before workout of the subject 211.

In the example illustrated in FIG. 7, regarding the first characteristic P1, a first time t1 when the pulse rate of the subject 211 decreases and reaches the first value V1 after increasing to reach the third value V3, and a second time t2 after an elapse of a predetermined first time r1 from the first time t1 are illustrated.

Here, in this embodiment, a state where the body is moving and the heart rate zone is at level 1 is used as the warming-up state, for example.

In the warming-up state, the subject 211 performs preparatory exercises, for example.

In this example, the second time t2 is used as the output timing of advice.

The content of the advice to be reported is what to be aware of in the next workout, for example.

In this example, the information processing device 1 uses pulse rate as an input, and uses a determination reference that pulse rates estimated warming-up have continued for a certain period.

Note that the timing before workout may be a timing immediately before workout, or other timings, for example.

Here, in the example illustrated in FIG. 7, the exercise intensity is used as an index, but as another example, instead of exercise intensity, the exercise amount may be used as an index, or a combination of exercise intensity and exercise amount may be used as an index. In addition, in this example, the first time r1 is a value greater than 0, but as another example, the first time r1 may be 0.

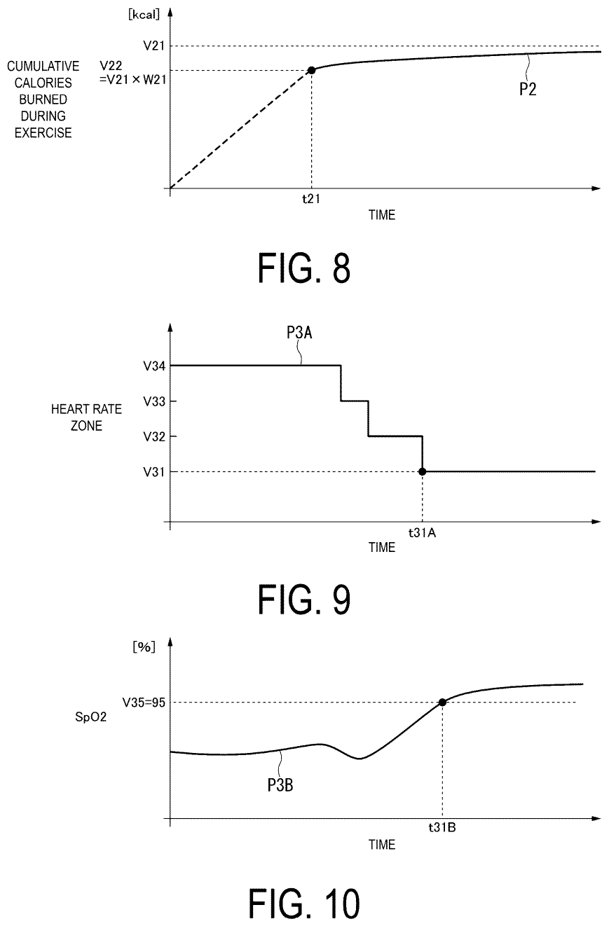

FIG. 8 is a graph illustrating an example of a tendency of a second pattern according to the embodiment.

In the graph, the abscissa indicates time, and the ordinate indicates cumulative calories burned during exercise [kcal].

In the graph, a second characteristic P2 representing a relationship between time and cumulative calories burned during exercise is illustrated.

In addition, in the graph, a twenty-first value V21, which is a target value of cumulative calories burned during exercise, and a twenty-the second value V22, which is smaller than the twenty-first value V21 and serves as a threshold value are illustrated. The twenty-the second value V22 corresponds to V21×W21, i.e., a result of multiplication of a predetermined twenty-first ratio value W21 by the twenty-first value V21.

Here, the twenty-first ratio value W21 is greater than 0 and smaller than 1.

In the second pattern, the output timing of advice is a timing when the subject 211 is performing workout and transitioning from an activity with high exercise intensity to an activity with low exercise intensity.

Here, the activity with high exercise intensity is performed within a predetermined period, for example.

In the example illustrated in FIG. 8, regarding the second characteristic P2, a twenty-first time t21 when the cumulative calories burned during exercise of the subject 211 increases and reaches the twenty-the second value V22 is illustrated.

In this example, the twenty-first time t21 is used as the output timing of advice.

The content of the advice to be reported is content to promote a change of the content of workout, for example.

In this example, the information processing device 1 uses calories burned during exercise as an input, and uses a determination reference that the value of the cumulative calories burned during exercise has reached a predetermined percentage of target calories.

Note that when determining the value of the cumulative calories burned during exercise, the cumulative value is reset to zero each time the subject 211 stops the exercise, for example.

In addition, the predetermined ratio is not limited, and may be 0.8 or the like, for example.

FIG. 9 is a graph illustrating an example of a tendency of a third pattern A according to the embodiment.

The third pattern A is an example of the third pattern.

In the graph, the abscissa indicates time, and the ordinate indicates a heart rate zone.

In the graph, a third A characteristic P3A representing a relationship between time and heart rate zone is illustrated.

In addition, in the graph, a thirty-first value V31, a thirty-second value V32, a thirty-third value V33, and a thirty-fourth value V34 representing heart rate zone are illustrated.

In this example, the thirty-first value V31 corresponds to the easy zone, the thirty-second value V32 to the fat burning zone, the thirty-third value V33 to the aerobic zone, and the thirty-fourth value V34 the anaerobic zone.

In the third pattern A, the output timing of advice is a timing after a workout of the subject 211 and after a rest of the subject 211. This timing is a timing when the subject 211 has had sufficient rest, for example.

In the example illustrated in FIG. 9, regarding the third A characteristic P3A, a thirty-first time t31A when the heart rate zone of the subject 211 decreases from the anaerobic zone and reaches the easy zone is illustrated.

In this example, the thirty-first time t31A is used as the output timing of advice.

The content of the advice to be reported is a review of the previous workout, for example.

In this example, the information processing device 1 uses heart rate zone as an input, and uses a determination reference that the level of heart rate zone has decreased and reached the easy zone.

Here, in the third pattern A, the thirty-first time t31A is set as the output timing of advice, but as another example, a time after an elapse of a predetermined time from the thirty-first time t31A may be set as the output timing of advice. In this case, for example, the predetermined time may be set variable in accordance with the heart rate zone value before the time t31A, and the value may be a maximum value, for example.

FIG. 10 is a graph illustrating an example of a tendency of a third pattern B according to the embodiment. The third pattern B is another example of the third pattern.

In the graph, the abscissa indicates time, and the ordinate indicates a value of SpO2 [%]. In the graph, a third B characteristic P3B representing a relationship between time and SpO 2 is illustrated.

In addition, in the graph, a thirty-fifth value V35, which is a predetermined SpO2 value and serves as a threshold value, is illustrated. The predetermined value is, for example, 95, but this is not limitative.

In the third pattern B, the output timing of advice is a timing after a workout of the subject 211 and after a rest of the subject 211. This timing is a timing when the subject 211 has had sufficient rest, for example.

In the example illustrated in FIG. 10, regarding the third B characteristic P3B, a thirty-first B time t31B when the SpO2 value of the subject 211 increases and reaches the thirty-fifth value V35 is illustrated.

In this example, the thirty-first B time t31B is used as the output timing of advice.

The content of the advice to be reported is a review of the previous workout, for example.

In this example, the information processing device 1 uses SpO2 as an input, and uses a determination reference that advice is reported when the SpO2 value is in a normal range. The normal range is 95 to 100 [%], for example.

Here, this example takes into account that blood oxygen may become deficient during exercise.

Here, in the third pattern B, the thirty-first B time t31B is set as the output timing of advice, but as another example, a time after an elapse of a predetermined time from the thirty-first B time t31B may be set as the output timing of advice. In this case, for example, the predetermined time may be set variable in accordance with the SpO2 value before the time t31B, and the value may be a minimum value, for example.

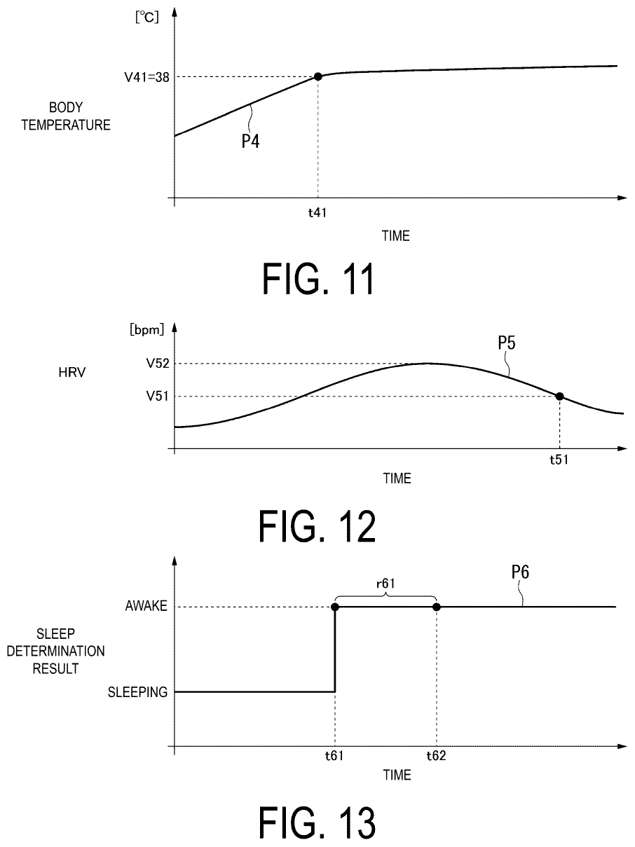

FIG. 11 is a graph illustrating an example of a tendency of a fourth pattern according to the embodiment.

In the graph, the abscissa indicates time, and the ordinate indicates a body temperature value [° C.].

In the graph, a fourth characteristic P4 representing a relationship between time and body temperature is illustrated.

In addition, in the graph, a forty-first value V41, which is a predetermined value of body temperature and serves as a threshold value, is illustrated. The predetermined value is, for example, 38, but this is not limitative.

In this example, the predetermined value corresponds to the upper limit value of the normal range of body temperature.

In the fourth pattern, the output timing of advice is a timing when an abnormality is detected in the biometric state of the subject 211.

In the example illustrated in FIG. 11, regarding the fourth characteristic P4, a forty-first B time t41 when the body temperature of the subject 211 has been abruptly increased to reach the forty-first value V41 and exceed the upper limit value of the normal range is illustrated.

In this example, the forty-first time t41 is used as the output timing of advice.

The content of the advice to be reported is a content to care the body, for example.

In this example, the information processing device 1 uses the body temperature as an input, and uses a determination reference that the body temperature has been abruptly increased and exceeded out of the normal range.

FIG. 12 is a graph illustrating an example of a tendency of a fifth pattern according to the embodiment.

In the graph, the abscissa indicates time, and the ordinate indicates an HRV value [bpm].

In the graph, a fifth characteristic P5 representing a relationship between time and HRV [bpm] is illustrated. In addition, in the graph, a fifty-first value V51, which is a predetermined value of HRV, and a fifty-second value V52, which is another predetermined value of HRV are illustrated. The fifty-second value V52 is greater than the fifty-first value V51.

In the fifth pattern, the output timing of advice is a timing when the subject 211 has returned to a normal state from a mindfulness state.

In the example illustrated in FIG. 12, regarding the fifth characteristic P5, a fifty-first time t51 when the HRV of the subject 211 increases to reach the fifty-second value V52, and then decreases to reach the fifty-first value V51 is illustrated.

In this example, the fifty-first time t51 is used as the output timing of advice.

The content of the advice to be reported is a content to promote mindfulness activities, for example.

In this example, the information processing device 1 uses HRV as an input, and uses a determination reference that the state is changed from a relaxed state to a neutral state, so as to be returned to the neutral state.

In general, mindfulness activities are those that regulate the state of mind through meditation and other means. Such activities result in a relaxed and drowsy state with high HRV values, which is referred to as mindfulness state.

FIG. 13 is a graph illustrating an example of a tendency of a sixth pattern according to the embodiment.

In the graph, the abscissa indicates time, and the ordinate indicates a sleep determination result.

In the graph, a sixth characteristic P6 representing a relationship between time and sleep determination result is illustrated.

In addition, in the graph, a sleeping state and an awake state are illustrated as a sleep determination result.

Here, the timing when the subject 211 becomes a sleeping state from an awake state corresponds to the timing when the subject 211 falls asleep.

In addition, the timing when the subject 211 becomes an awake state from a sleeping state corresponds to the timing when the subject 211 wakes up.

In the sixth pattern, the output timing of advice is a timing when the head awakens to a certain degree after the subject 211 wakes up.

In the example illustrated in FIG. 13, regarding the sixth characteristic P6, a sixty-second time t62 after an elapse of a predetermined sixty-first time r61 from a sixty-first time t61 when the subject 211 becomes an awake state from a sleeping state is illustrated. That is, the sixty-first time r61 represents a predetermined elapsed time from a wake-up timing.

In this example, the sixty-second time t62 is used as the output timing of advice.

The content of the advice to be reported is a suggestion of something to be conscious of for the day, for example.

In this example, the information processing device 1 uses the sleep quality of the wake-up timing and a timing immediately before the wake-up timing as an input, and uses a determination reference that a predetermined time has elapsed from the wake-up timing. Here, the predetermined time is a sixty-first time r61 and may change depending on the sleep quality immediately before the subject 211 wakes up, for example. As a specific example, the predetermined may be set to time 10 minutes when the sleep quality immediately before the subject 211 wakes up is REM sleep, and the predetermined time may be set to 20 minutes when the sleep quality immediately before the subject 211 wakes up is non-REM sleep. Note that in this example, the information processing device 1 determines the sleep quality when the subject 211 is sleeping.

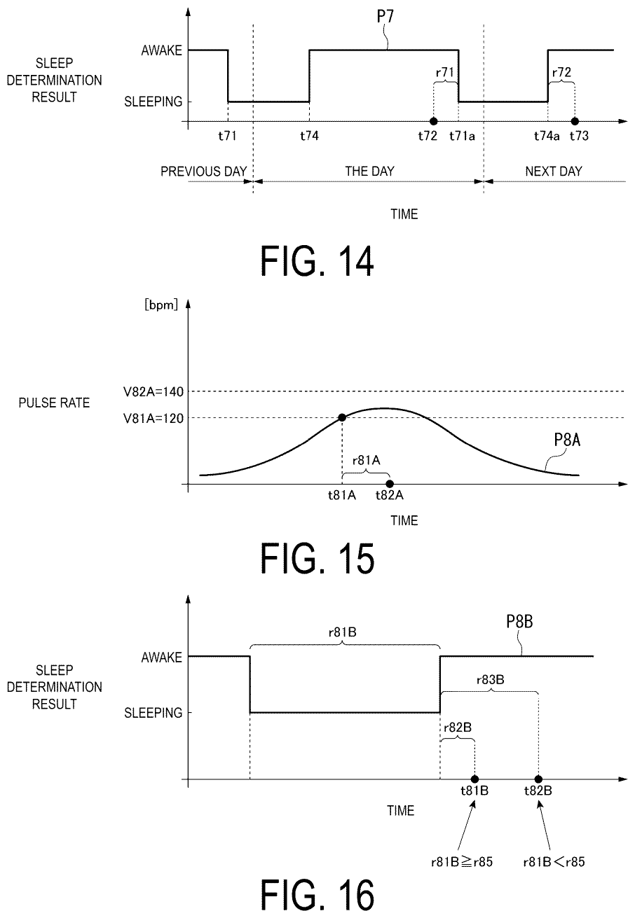

FIG. 14 is a graph illustrating an example of a tendency of a seventh pattern according to the embodiment.

In the graph, the abscissa indicates time, and the ordinate indicates a sleep determination result.

In the graph, a seventh characteristic P7 representing a relationship between time and sleep determination result is illustrated.

In addition, in the graph, a sleeping state and an awake state are illustrated as a sleep determination result.

In the seventh pattern, the output timing of advice is a timing before the subject 211 falls asleep, or a timing after the subject 211 after wakes up.

In the example illustrated in FIG. 14, regarding the seventh characteristic P7, a seventy-first time t71 when the subject 211 becomes a sleeping state from an awake state on the previous day, a seventy-second time t72 corresponding to a time obtained by subtracting a seventy-first time r71 from the same time as the seventy-first time t71 on the day, and a seventy-third time t73 when a seventy-second time r72 has elapsed from the time when the subject 211 becomes an awake state from a sleeping state on the next day are illustrated.

Here, the seventy-first time r71 is a predetermined time and may be 10 minutes or the like, for example. In addition, the seventy-second time r72 is a predetermined time and may be 10 minutes or the like, for example.

Note that “the day” illustrated in FIG. 14 is the previous day of “the next day”. In addition, “the next day” illustrated in FIG. 14 is the day after the “the day”. In addition, “the day” illustrated in FIG. 14 is the next day of the “the previous day”.

In this example, the information processing device 1 acquires the HRV of the subject 211 and the like, and determines the output timing of advice on the basis of the HRV and the like.

For example, when the HRV is lower than a predetermined value, the information processing device 1 sets the seventy-second time t72 earlier than the seventy-first time t71a of the day as the output timing of advice. Alternatively, the information processing device 1 sets the seventy-third time t73 later than a seventy-fourth time t74a of the next day as the output timing of advice.

Here, the seventy-first time t71a of the day is the same time as the seventy-first time t71, which is the sleep time of the previous day, for example.

In addition, the seventy-fourth time t74a of the next day is the same time as the seventy-fourth time t74, which is the waking-up time of the day, for example.

Note that the seventy-first time t71a is an example of the first timing that takes into account the bedtime up to the previous day alone, and the seventy-second time t72 is an example of the second timing that takes into account one or both of the exercise and HRV of the day for the first timing.

In addition, the seventy-fourth time t74a is an example of the first timing that takes into account the wake-up time up to the day alone, and the seventy-third time t73 is an example of the second timing that takes into account one or both of HRV, and sufficiency of sleep time for the exercise of the day for the first timing, for example.

Note that the configuration of setting the second timing in consideration of HRV and the like for the first timing is not limited to this example, and various configurations may be used.

As described above, in this example, the seventy-second time t72 or the seventy-third time t73 are used as the output timing of advice.

The content of the advice to be reported is a review of the day's activity, for example.

In this example, the information processing device 1 uses the bedtime timing of the previous day and the HRV before falling asleep of the day as an input, stores the bedtime timing of the previous day, and uses a determination reference for determining the HRV state at timing a predetermined time earlier than the bedtime timing of the day.

Note that the seventy-third time t73 in the seventh pattern may be the same as the sixty-second time t62 in sixth pattern, for example.

In this example, when the HRV of the subject 211 is low, the brain of the subject 211 is considered awake, whereas when the HRV of the subject 211 is high, the brain of the subject 211 is considered not awake.

FIG. 15 is a graph illustrating an example of a tendency of an eighth pattern A according to the embodiment. The eighth pattern A is an example of a pattern for determining the required sleep time in the eighth pattern.

In the graph, the abscissa indicates time, and the ordinate indicates a pulse rate value [bpm]. In the graph, an eighth A characteristic P8A representing a relationship between time and pulse rate is illustrated.

In addition, in the graph, an eighty-first A value V81A and an eighty-second A value V82A, which are values representing pulse rates and serve as threshold values, are illustrated.

Here, the eighty-second A value V82A is greater than the eighty-first A value V81A.

As an example, the eighty-first A value V81A is 120 [bpm], and the eighty-second A value V82A is 140 [bpm].

In this example, the information processing device 1 sets the required sleep time based on whether the maximum heart rate strength has exceeded a set value. In this setting, for example, any of two or more times set in advance may be set as the required sleep time.

As a specific example, the information processing device 1 sets the required sleep time to seven hours when the maximum heart rate strength of the subject 211 in a predetermined period is smaller than the eighty-first A value V81A, sets the required sleep time to eight hours when the maximum heart rate strength is greater than the eighty-first A value V81A but is smaller than the eighty-second A value V82A, and sets the required sleep time to a predetermined time longer than eight hours when the maximum heart rate strength is greater than the eighty-second A value V82A. The predetermined time is nine hours or the like, for example.

In the example illustrated in FIG. 15, for convenience of description, when it is assumed that the predetermined period is a period from an eighty-first time t81A to an eighty-second time t82A, the eighth A characteristic PSA is greater than the eighty-first A value V81A but is smaller than the eighty-second A value V82A, and as such the required sleep time is set to eight hours. Here, in the example illustrated in FIG. 15, the length of the predetermined period is the eighty-first time r81A.

Note that as another example, the information processing device 1 may determine and set a continuously changeable required sleep time by calculating a predetermined computation expression for calculating the required sleep time with the maximum heart rate strength as a parameter.

In this example, the information processing device 1 uses pulse rate as an input, and uses a determination reference for determining the maximum value of pulse rate.

Here, in general, when there is no arrhythmia, the heart rate and the pulse rate coincide with each other, and the maximum heart rate strength coincides with the maximum value of pulse rate.

Note that as another example, heart rate and the like may be used instead of pulse rate.

FIG. 16 is a graph illustrating an example of a tendency of an eighth pattern B according to the embodiment. The eighth pattern B is an example of a pattern representing the output timing of advice in the eighth pattern.

In the graph, the abscissa indicates time, and the ordinate indicates a sleep determination result.

In the graph, an eighth B characteristic P8B representing a relationship between time and sleep determination results is illustrated. In addition, in the graph, a sleeping state and an awake state are illustrated as a sleep determination result.

In the eighth pattern B, the output timing of advice is a timing when a predetermined time corresponding to the sleep time has elapsed after the subject 211 wakes up.

In the example illustrated in FIG. 16, regarding the eighth B characteristic P8B, the sleep time of the subject 211 is an eighty-first B time r81B, and an eighty-first B time t81B after an elapse of an eighty-second B time r82B from the time when the subject 211 becomes an awake state from a sleeping state, and an eighty-second B time t82B after an elapse of an eighty-third B time r83B from the time when the subject 211 becomes an awake state from a sleeping state are illustrated.

Here, the eighty-third B time r83B is greater than the eighty-second B time r82B. That is, the eighty-second B time t82B is a timing later than the eighty-first B time t81B. The eighty-third B time r83B and the eighty-second B time r82B may be set to any time.

In addition, the eighty-first B time r81B represents the sleep time of the subject 211.

For example, the predetermined period before sleep in the eighth pattern B illustrated in FIG. 16 is the period of the eighth pattern A illustrated in FIG. 15. The predetermined period may be a time corresponding to one day, for example.

In this example, when the eighty-first B time r81B, which is the sleep time of the subject 211, is equal to or greater than an eighty-fifth time r85, which is a predetermined threshold value, the information processing device 1 sets the eighty-first B time t81B as the output timing of advice, whereas when the eighty-first B time r81B, which is the sleep time of the subject 211, is smaller than the eighty-fifth time r85, which is a predetermined threshold value, the information processing device 1 sets the eighty-second B time t82B as the output timing of advice.

Here, the eighty-fifth time r85 is the required sleep time set based on the eighth pattern A illustrated in FIG. 15, and is, for example, the sleep time considered sufficient for recovery from fatigue due to an exercise in a predetermined period before sleep. The predetermined period is one day, for example.

As a specific example, in the case where the exercise intensity of the subject 211 before sleep is high and needs eight hours sleep, when sleep hours are eight hours, the information processing device 1 reports advice at the eighty-first B time t81B that is a timing when a predetermined time has elapsed after the subject 211 wakes up, whereas when sleep hours are shorter than eight hours, the information processing device 1 reports advice at the eighty-second B time t82B that is a timing when a time longer than the predetermined time has elapsed after the subject 211 wakes up.

In this manner, in this example, the eighty-first B time t81B or the eighty-second B time t82B is used as the output timing of advice.

The content of the advice to be reported is a content related to the activity including the next day's exercise, for example.

In this example, the information processing device 1 uses the sleep time as an input, and uses a determination reference regarding whether the sleep time is the required sleep time or longer.

Here, in the example illustrated in FIG. 15, the exercise intensity is used as an index, but as another example, the exercise amount may be used as an index instead of exercise intensity, or a combination of exercise intensity and exercise amount may be used as an index.

In addition, in the example illustrated in FIG. 16, the eighty-second B time r82B defining the eighty-first B time t81B is a value greater than 0, but as another example, the eighty-second B time r82B may be b 0.

Next, a specific example of a process performed by the information processing device 1 is described.

In the information processing device 1, the acquisition unit 111 acquires the biometric information of the subject 211. The determination unit 112 determines the state of the subject 211 from the biometric information. The generation unit 113 generates advice corresponding to the state of the subject 211. The output unit 12 outputs the generated advice.

In this case, the output unit 12 changes the output timing of advice in accordance with the biometric information.

Thus, the information processing device 1 can output advice at a timing corresponding to the biometric information of the subject 211.

Here, the output timing of advice may be determined with reference to information other than biometric information, for example.

In addition, the content of advice is not limited, and various content may be used. Note that in the example illustrated in FIG. 1, the timing determination unit 114 determines the output timing of advice, and the output unit 12 outputs the advice at that timing as an example, but the function of the timing determination unit 114 may be included in the output unit 12.

In the information processing device 1, the biometric information includes at least one of pulsation, SpO2, and body temperature.

Thus, the information processing device 1 can determine the exercise state of the subject 211 by acquiring information about any of the pulsation, SpO2, and body temperature of the subject 211, for example;

In the information processing device 1, the acquisition unit 111 acquires the exercise intensity on the basis of biometric information. When the exercise intensity equal to or greater than the first threshold value and smaller than the second threshold value, the determination unit 112 determines that the state of the subject 211 is the first exercise state. The generation unit 113 generates advice on the exercise on the basis of the first exercise state. The output unit 12 outputs the advice on the exercise after a predetermined first time has elapsed after it is determined that the state is the first exercise state.

Thus, the information processing device 1 can report advice at a timing after warming-up and before workout. For example, the information processing device 1 can generate and output advice on the next workout when the subject 211 has performed warming-up, and can present advice on what the subject 211 should do next.

Here, in this example, the first pattern illustrated in FIG. 7 may be applied, for example.

For example, the exercise intensity is determined on the basis of pulse rate, heart rate zone or the like.

The first exercise state is a warming-up state, for example.

In the example illustrated in FIG. 7, the first value V1 is an example of the first threshold value, and the second value V2 is an example of the second threshold value.

In the example illustrated in FIG. 7, the first time r1 is an example of the first time. Note that first time is a value greater than 0.

In the information processing device 1, the predetermined first time for the first exercise intensity with the maximum value of the exercise intensity close to the first threshold value is shorter than the predetermined first time for the second exercise intensity with the maximum value of the exercise intensity close to the second threshold value.

Thus, the information processing device 1 can change the output timing of advice in accordance with the exercise intensity, and can present advice at an appropriate timing, for example. For example, when the maximum value of the exercise intensity is small, the rest time after the warming-up before advice is shorter, and when the maximum value of the exercise intensity is large, the rest time after the warming-up before advice is longer.

Here, in this example, the first pattern illustrated in FIG. 7 may be applied, for example.

In the example illustrated in FIG. 7, the third value V3 is an example of the maximum value of the exercise intensity.

In this embodiment, the timing determination unit 114 changes the first time in accordance with the maximum value of the exercise intensity.

In the information processing device 1, when the exercise intensity is equal to or greater than the second threshold value, the determination unit 112 determines that the state of the subject 211 is the second exercise state. The generation unit 113 generates advice on an activity including an exercise on the basis of the second exercise state of the subject 211. The output unit 12 outputs advice on the activity at the second timing earlier or later than the first timing in accordance with the first exercise state and the second exercise state.

Thus, the information processing device 1 can change the timing of the advice on the day's activity in accordance with the load state during the exercise of the subject 211, and, for example, can present advice on the day's activity in accordance with the load of the subject 211.

The load state during the exercise is the load state in the first exercise state, and the load state in the second exercise state, for example. The load state may be determined on the basis of the time, strength or the like, for example.

Here, in this example, the seventh pattern illustrated in FIG. 14 may be applied, for example.

The second exercise state is a workout state, for example.

In the example illustrated in FIG. 14, the sleep state of the subject 211 of the day has a value corresponding to the first exercise state and the second exercise state, that is, a value corresponding to a state of the combination of warming-up and workout.

Note that the activity is not limited, and may include various activities such as the exercise performed by the subject 211, sleep, and meal, for example.

In the information processing device 1, advice on the activity includes advice to the subject 211 to promote sleep.

Thus, in the information processing device 1, advice on the activity may include advice on sleep, and for example, advice on sleep may be presented in accordance with the load of the subject 211.

Here, in this example, the seventh pattern illustrated in FIG. 14 may be applied, for example.

In the information processing device 1, the detection unit detects the movement of the subject 211. The storage unit 14 stores information about sleep of the subject 211. The acquisition unit 111 acquires the bedtime or wake-up time of the subject 211 on the basis of the movement and biometric information of the subject 211. The storage unit 14 stores the bedtime or wake-up time of the subject 211 up to the previous day. The second timing is a timing corresponding to the bedtime or wake-up time of the subject 211 up to the previous day.

Thus, in the information processing device 1, the second timing is a timing based on the usual bedtime or wake-up time of the subject 211, and for example, the information processing device 1 can output advice earlier than the bedtime that is the usual sleep timing of the subject 211, or output advice later than the usual wake-up time of the subject 211.

Here, in this embodiment, the movement sensor C3 is an example of the detection unit that detects the movement of the subject 211.

In addition, the bedtime or wake-up time of the subject 211 up to the previous day may be the bedtime or wake-up time of the previous day or another day, or an average value of the bedtime or wake-up time of a plurality of days up to the previous day and the like, for example.

Here, in this example, the seventh pattern illustrated in FIG. 14 may be applied, for example.

In the information processing device 1, the detection unit detects the movement of the subject 211. The storage unit 14 stores information about sleep of the subject 211. When the exercise intensity is equal to or greater than the second threshold value, the determination unit 112 determines that the state of the subject 211 is the second exercise state. The generation unit 113 generates advice on the activity including the next day's exercise on the basis of the first exercise state and the second exercise state. The acquisition unit 111 acquires the wake-up time of the subject 211 and the sleep time of the subject 211 on the basis of the movement and biometric information of the subject 211. The determination unit 112 determines whether the sleep time is sufficient for the exercise intensity for the day including the exercise intensity in the first exercise state and the second exercise state. When it is determined that the sleep time is insufficient, the output unit 12 outputs advice on the activity including the next day's exercise at a fourth timing later than a third timing when the sleep time is determined to be sufficient.

Thus, the information processing device 1 can change the output timing of advice upon wake-up in accordance with the exercise intensity and sleep time, and for example, can delay the output timing of advice when the sleep time is insufficient for the exercise intensity.

Here, in this example, for example, the eighth pattern illustrated in FIGS. 15 and 16 may be applied.

In the example illustrated in FIG. 15, the required sleep time is determined for the exercise intensity for the day including the exercise intensity of the warming-up and workout state of one day before the next day.

In the example illustrated in FIG. 16, in the next day, the eighty-first B time t81B is an example of the third timing, and the eighty-second B time t82B is an example of the fourth timing.

In the information processing device 1, when the exercise intensity is equal to or greater than the second threshold value, the determination unit 112 determines that the state of the subject 211 is the second exercise state. The generation unit 113 generates advice to promote different exercises on the basis of the second exercise state of the subject 211. When the exercise intensity of the second exercise state satisfies a predetermined strength, the output unit 12 outputs advice to promote different exercises.

Thus, the information processing device 1 can present advice to change the exercise at an appropriate timing for the exercise state of the subject 211, and for example can present advice to reduce the exercise intensity at the time when a predetermined exercise intensity has reached in the workout.

Here, in this example, for example, a pattern using exercise intensity may be applied instead of calories burned during exercise in the second pattern illustrated in FIG. 8.

In the information processing device 1, the generation unit 113 generates advice on review of the exercise on the basis of the first exercise state and the second exercise state. When it is determined that the exercise intensity becomes an intensity smaller than the first threshold value from an intensity equal to or greater than the second threshold value, the output unit 12 outputs advice on review of the exercise after a predetermined second time has elapsed.

Thus, the information processing device 1 can present advice on the overall exercise at an appropriate timing, and for example can present advice on the overall exercise after a workout.

Here, in this example, in the third pattern A illustrated in FIG. 9 a pattern in which the time after an elapse of the second time from a thirty-first A time is set to the output timing of advice may be applied, for example. Note that the second time is a value greater than 0, but may be applied to the third pattern A in the case where the second time is 0.

Likewise, in this example, in the third pattern B illustrated in FIG. 10 a pattern in which the time after an elapse of the second time from a thirty-first B time is set to the output timing of advice may be applied, for example. Note that the second time is a value greater than 0, but may be applied to the third pattern B in the case where the second time is 0.

In the information processing device 1, the predetermined second time for the case where the exercise intensity equal to or greater than the second threshold value and equal to or greater than the third threshold value is acquired by the acquisition unit 111 is longer than the predetermined second time for the case where the exercise intensity equal to or greater than the second threshold value and equal to or greater than the third threshold value is not acquired by the acquisition unit 111.

Thus, the information processing device 1 can present the advice at an appropriate timing, and for example can change the output timing of the advice in accordance with the exercise intensity.

Here, in this example, for example, in the third pattern A illustrated in FIG. 9 a pattern in which the time after an elapse of the second time from the thirty-first time t31A is set to the output timing of advice may be applied. In this case, for example, the thirty-fourth value V34 is an example of the third threshold value, but the third threshold value may be other values.

Likewise, in this example, in the third pattern B illustrated in FIG. 10 a pattern in which the time after an elapse of the second time from the thirty-first B time t31B is set to the output timing of advice may be applied, for example.

In the information processing device 1, the acquisition unit 111 acquires the exercise amount on the basis of the biometric information. When the exercise amount is the first exercise amount, the determination unit 112 determines that the state of the subject 211 is the first exercise state. The generation unit 113 generates advice on the exercise on the basis of the first exercise state. The output unit 12 outputs the advice on the exercise when a predetermined third time has elapsed after it is determined that the state is the first exercise state.

Thus, for example, the information processing device 1 can generate and output advice on the next workout when the subject 211 has performed warming-up, and can present advice on what the subject 211 should do next.

Here, in this example, in the first pattern illustrated in FIG. 7 a pattern that uses exercise amount instead of pulse rate may be applied, for example. The exercise amount may be calories burned during exercise, for example.

Note that the first exercise amount of the case where the exercise amount is used as in this example may be set to a value for determining the warming-up state as with the first threshold value in the case where exercise intensity is used, for example.

In addition, the predetermined third time of the case where the exercise amount is used as in this example may be set to various values as with the predetermined first time of the case where the exercise intensity is used, for example.

In addition, as another example, a configuration that uses a combination of the exercise amount and the exercise intensity may be employed.

In the information processing device 1, the determination unit 112 determines that the state of the subject 211 is the second exercise state when the exercise amount is equal to or greater than the second exercise amount, which is greater than the first exercise amount. The generation unit 113 generates advice on an activity including an exercise on the basis of the second exercise state of the subject 211. The output unit 12 outputs the advice on activity at a sixth timing earlier than a fifth timing in accordance with the first exercise state and the second exercise state.

Thus, the information processing device 1 can change the timing of the advice on the day's activity in accordance with the load state during the exercise of the subject 211, and, for example, can present advice on the day's activity in accordance with the load of the subject 211.

Here, in this example, the seventh pattern illustrated in FIG. 14 may be applied, for example.

Note that the second exercise amount of the case where the exercise amount is used as in this example may be set to a value for determining workout state as with the second threshold value of the case where the exercise intensity is used, for example.

In addition, the fifth timing and sixth timing of the case where the exercise amount is used as in this example may be set to various values as with the first timing and second timing of the case where the exercise intensity is used, for example.

In addition, as another example, a configuration that uses a combination of the exercise amount and the exercise intensity may be employed.

Here, the case using the exercise intensity and the case using the exercise amount are different in the computation expression for calculating the value for specifying the first exercise state and the second exercise state, for example. The computation expression is a computation expression whose parameter is exercise intensity, or a computation expression whose parameter is exercise amount, or as another example, a computation expression whose parameter is both exercise intensity and exercise amount, for example. Note that these computation expressions may include parameters different from exercise intensity and exercise amount, for example.

In the information processing device 1, advice on the activity includes advice to the subject 211 to promote sleep.

Thus, in the information processing device 1, advice on the activity may include advice on sleep, and for example, advice on sleep may be presented in accordance with the load of the subject 211.

Here, in this example, the seventh pattern illustrated in FIG. 14 may be applied, for example.

In the information processing device 1, the detection unit detects the movement of the subject 211. The storage unit 14 stores information about sleep of the subject 211. The acquisition unit 111 acquires the bedtime or wake-up time of the subject 211 on the basis of the movement and biometric information of the subject 211. The storage unit 14 stores the bedtime or wake-up time of the subject 211 up to the previous day. The sixth timing is a timing corresponding to the bedtime or wake-up time of the subject 211 up to the previous day.

Thus, in the information processing device 1, the sixth timing is a timing based on the usual bedtime or wake-up time of the subject 211, and for example, the information processing device 1 can output advice earlier than the bedtime that is the usual sleep timing of the subject 211, or output advice later than the usual wake-up time of the subject 211.

Here, in this example, the seventh pattern illustrated in FIG. 14 may be applied, for example.

In the information processing device 1, the detection unit detects the movement of the subject 211. The storage unit 14 stores information about sleep of the subject 211. When the exercise amount is equal to or greater than the second exercise amount, the determination unit 112 determines that the state of the subject 211 is the second exercise state. The generation unit 113 generates advice on the activity including the next day's exercise on the basis of the first exercise state and the second exercise state. The acquisition unit 111 acquires the wake-up time of the subject 211 and the sleep time of the subject 211 on the basis of the movement and biometric information of the subject 211. The determination unit 112 determines whether the sleep time is sufficient for the day's exercise amount including the exercise amount in the first exercise state and the second exercise state. When it is determined that sleep time is insufficient, the output unit 12 outputs the advice on the activity including the next day's exercise at an eighth timing later than a seventh timing on the basis of the wake-up time of the subject 211 up to the previous day.

Thus, the information processing device 1 can change the output timing of advice upon wake-up in accordance with the exercise intensity and sleep time. For example, the information processing device 1 can delay the output timing of advice when the sleep time is insufficient for the exercise amount.

Here, in this example, in the eighth pattern A in the eighth pattern illustrated in FIGS. 15 and 16 a pattern that uses exercise amount instead of pulse rate may be applied, for example. The exercise amount may be calories burned during exercise, for example.

In addition, the seventh timing and eighth timing of the case where the exercise amount is used as in this example may be set to various values as with the third timing and fourth timing of the case where the exercise intensity is used, for example.

In addition, as another example, a configuration that uses a combination of the exercise amount and the exercise intensity may be employed.

In the information processing device 1, the determination unit 112 determines that the state of the subject 211 is the second exercise state when the exercise amount is equal to or greater than the second exercise amount, which is greater than the first exercise amount. The generation unit 113 generates advice to promote different exercises on the basis of the second exercise state of the subject 211. When the exercise amount of the second exercise state satisfies the predetermined exercise amount, the output unit 12 outputs advice to promote different exercises.

Thus, the information processing device 1 can present advice to change the exercise at an appropriate timing for the exercise state of the subject 211, and for example can present advice to reduce the exercise amount at the time when a predetermined exercise amount has reached in the workout.

Here, in this example, the second pattern illustrated in FIG. 8 may be applied, for example.

Note that the predetermined exercise amount of the case where the exercise amount is used as in this example may be set to various values as with the predetermined strength of the case where the exercise intensity is used, for example.

In addition, as another example, a configuration that uses a combination of the exercise amount and the exercise intensity may be employed.

In the information processing device 1, the generation unit 113 generates advice on review of the exercise on the basis in the first exercise state and the second exercise state. When it is determined that the exercise amount has changed from the exercise amount of the second exercise state to an exercise amount equal to or smaller than the exercise amount in the first exercise state, the output unit 12 outputs advice on review of the exercise after an elapse of a predetermined fourth time.

Thus, the information processing device 1 can present advice on the overall exercise at an appropriate timing, and for example can present advice on the overall exercise after a workout.

Here, in this example, in the third pattern A illustrated in FIG. 9 a pattern that uses exercise amount instead of pulse rate may be applied, for example. The exercise amount may be the calories burned during exercise per predetermined unit time, for example. The predetermined unit may be any value.

In this case, in this example, in the third pattern A illustrated in FIG. 9 a pattern in which the time after an elapse of the fourth time from the thirty-first A time is set to the output timing of advice may be applied, for example. Note that the fourth time is a value greater than 0, but may be applied to the third pattern A in the case where the fourth time is 0.

Likewise, in this example, in the third pattern B illustrated in FIG. 10 a pattern that uses exercise amount instead of SpO2 may be applied, for example. The exercise amount may be the calories burned during exercise per predetermined unit time, for example. The predetermined unit may be any value.

In this case, in this example, in the third pattern B illustrated in FIG. 10 a pattern in which the time after an elapse of the fourth time from the thirty-first B time is set to the output timing of advice may be applied, for example. Note that the fourth time is a value greater than 0, but may be applied to the third pattern B in the case where the fourth time is 0.

In the information processing device 1, the predetermined fourth time corresponding to a first total exercise amount in the first exercise state and the second exercise state is longer than the predetermined fourth time corresponding to the second total exercise amount smaller than the first total exercise amount.

Thus, the information processing device 1 can present the advice at an appropriate timing, and for example can change the output timing of the advice in accordance with the exercise amount.

Here, in this example, in the third pattern A illustrated in FIG. 9 a pattern in which the time after an elapse of the fourth time from the thirty-first time t31A is set to the output timing of advice may be applied, for example. Note that the first total exercise amount and second total exercise amount of the case where the exercise amount is used as in this example may be set to various values as with the third threshold value of the case where the exercise intensity is used, for example.

Likewise, in this example, in the third pattern B illustrated in FIG. 10 a pattern in which the time after an elapse of the fourth time from the thirty-first B time t31B is set to the output timing of advice may be applied, for example.

In addition, as another example, a configuration that uses a combination of the exercise amount and the exercise intensity may be employed.

In the information processing device 1, the biometric information includes information about pulsation. The acquisition unit 111 acquires data related to heart rate variability from the biometric information. The output unit 12 outputs advice on the activity when there is a predetermined change in the data related to heart rate variability.

Thus, the information processing device 1 can be set to not output advice when the head of the subject 211 is not awake, and can change the output timing of advice such as upon wake-up so as to output advice when the head of the subject 211 is in an awake state on the basis of HRV or the like, for example.

Here, in this example, for example, the fifth pattern illustrated in FIG. 12 or the sixth pattern illustrated in FIG. 13 may be applied.

Note that the data related to heart rate variability is calculated in a time domain or a frequency domain, for example. The predetermined change may be a state of sympathetic hyperactivity, for example.

A state of sympathetic hyperactivity may be determined from any of the following first hyperactivity example and second hyperactivity example, or their combinations.

The first hyperactivity example occurs when the variability in the intervals between heart rates is smaller than a predetermined value, such as when the RMSSD (Root Mean Square of Successive Differences of RR intervals) or the SDNN (Standard Deviation of Normal-to-Normal intervals) is low.

The second hyperactivity example occurs when the ratio of the frequency components of heart rates is smaller than a predetermined value, such as when the LF/HF ratio, which represents the balance between the sympathetic nervous system (LF: Low Frequency) and the parasympathetic nervous system (HF: High Frequency), is high, or when the LF value is high.

As described above, in the information processing device 1 according to this embodiment, the output timing of advice to the subject 211 can be changed in accordance with the biometric information of the subject 211, and thus advice can be output at a suitable timing in accordance with the biometric state of the subject 211, for example.

In the information processing device 1 according to this embodiment, advice is presented to the subject 211 at an appropriate timing by determining the exercise state of the subject 211 on the basis of the biometric information acquired from the subject 211, generating advice on the basis of the exercise state of the subject 211, and changing the output timing of advice to the subject 211 in accordance with the biometric information, for example.

As a specific example, the information processing device 1 according to this embodiment estimates a state suitable for acquiring advice for the subject 211 on the basis of the biometric information of the subject 211 during exercise or in other periods, and outputs advice at a timing when the state is the estimated state. In this manner, in the information processing device 1 according to this embodiment, the subject 211 can listen to advice in an easy-to-hear manner, and thus the exercise of the subject 211 can be supported, for example. In addition, the information processing device 1 according to this embodiment can present advice corresponding to the motivation of the subject 211, for example.

Here, in a configuration of acquiring only the body movement information of the player and the moving state information of the equipment and determining the timing of advice on the basis of the movement of the player alone as in known techniques, if the movement of the player is the same, advice is output at uniform timing regardless of the mental and physical states of the player, and consequently the output timing of advice may not be optimum.

In contrast, in this embodiment, advice is output in a state where the mental and physical states of the subject 211 are suitable for acquisition of advice, and thus the subject 211 can easily accept the advice, for example. Note that the mental and physical states of the subject 211 may include a variety of states, such as good, normal, or poor, for example.

A program for implementing the function of a given component in a given device described above may be recorded on a computer-readable recording medium, and executed by reading the program by a computer system. The “computer system” as used herein is assumed to include hardware such as an operating system or peripheral devices. The “computer-readable recording medium” is a storage device such as a portable medium such as a flexible disk, a magneto-optical disk, a read only memory (ROM), a compact disc (CD)-ROM, and a hard disk built into a computer system. The “computer-readable recording medium” is assumed to include a medium that holds a program for a certain period of time, such as a volatile memory provided inside of a computer system serving as a server or a client when the program is transmitted via a network such as the Internet or a communication line such as a telephone line. The volatile memory may be, for example, a random access memory (RAM). The recording medium may be a non-transitory recording medium.

The program described above may be transmitted to another computer system from a computer system storing this program in a storage device or the like via a transmission medium or transmission waves in a transmission medium. The “transmission medium” from which the program is transmitted refers to a medium having a function of transmitting information, such as a network such as the Internet or a communication line such as a telephone line.

The program described above may be a program for realizing a part of the above-described functions. The program described above may be a so-called difference file, which can realize the above-described functions in combination with a program already recorded in the computer system. The difference file may be called a difference program.

The function of a given component in a given device described above may be implemented by a processor. Each of the processes in the embodiment may be realized by a processor that operates on the basis of information such as a program and a computer-readable recording medium that stores information such as a program. In the processor, the function of each unit may be realized by individual hardware, or the function of each unit may be realized by integrated hardware. The processor may include hardware, and the hardware may include at least one of a circuit for processing digital signals and a circuit for processing analog signals. The processor may be configured using one of or both of one or more circuit devices or one or more circuit elements mounted on a circuit board. For the circuit device, an integrated circuit (IC) or the like may be used, and for the circuit element, a resistor, a capacitor, or the like may be used.

The processor may be a CPU. However, the processor is not limited to the CPU, and it may be possible to use various processors such as a graphics processing unit (GPU) or a digital signal processor (DSP). The processor may be a hardware circuit using an application-specific integrated circuit (ASIC). The processor may be configured by a plurality of CPUs or may be configured by a hardware circuit including a plurality of ASICs. The processor may be configured by a combination of a plurality of CPUs and a hardware circuit including a plurality of ASICs. The processor may include one or more of an amplifier circuit, a filter circuit, and the like for processing analog signals.

The above embodiments have been described in detail with reference to the drawings. Specific configurations are not limited to these embodiments, but include designs, etc., to the extent that they do not depart from the gist of this disclosure.

### Supplementary Notes

The following are configuration example 1 to configuration example 22.

The lower configuration examples may or may not be applied to the upper configuration examples.

The lower configuration examples applicable to any of the two or more upper configuration examples may be applied to any of the two or more upper configuration examples, and furthermore, when two or more applicable examples occur in this way, configuration examples even lower than the lower configuration examples concerned may be applied to any of these two or more applicable examples.

### Configuration Example 1

An information processing device including an acquisition unit configured to acquire biometric information of a subject, a determination unit configured to determine a state of the subject from the biometric information, a generation unit configured to generate advice corresponding to the state of the subject, and an output unit configured to output the advice generated, in which the output unit changes an output timing of the advice in accordance with the biometric information.

### Configuration Example 2

The information processing device according to Configuration Example 1, in which the biometric information includes at least any of pulsation, SpO2, and body temperature.

Configuration Example 3

The information processing device according to Configuration Example 1 or Configuration Example 2, in which the acquisition unit acquires an exercise intensity based on the biometric information, when the exercise intensity is equal to or greater than a first threshold value and is smaller than a second threshold value, the determination unit determines that the state of the subject is a first exercise state, the generation unit generates advice on an exercise based on the first exercise state, and the output unit outputs the advice on the exercise after an elapse of a predetermined first time after it is determined that the state is the first exercise state.

### Configuration Example 4

The information processing device according to Configuration Example 3, in which the predetermined first time of a case where a maximum value of the exercise intensity is a first exercise intensity close to the first threshold value is shorter than the predetermined first time of a case where the maximum value of the exercise intensity is a second exercise intensity close to the second threshold value.

### Configuration Example 5

The information processing device according to Configuration Example 4, in which when the exercise intensity is equal to or greater than the second threshold value, the determination unit determines that the state of the subject is a second exercise state, the generation unit generates advice on an activity including an exercise based on the second exercise state of the subject, and the output unit outputs the advice on the activity at a second timing earlier or later than the first timing in accordance with the first exercise state and the second exercise state.

### Configuration Example 6

The information processing device according to Configuration Example 5, in which the advice on the activity includes advice to the subject to promote sleep.

### Configuration Example 7

The information processing device according to Configuration Example 6, further including a detection unit configured to detect a movement of the subject, and a storage unit configured to store information about sleep of the subject, in which the acquisition unit acquires a bedtime or a wake-up time of the subject based on the movement and the biometric information of the subject, the storage unit stores the bedtime or the wake-up time of the subject up to the previous day, and the second timing is a timing corresponding to the bedtime or the wake-up time of the subject up to the previous day.

### Configuration Example 8

The information processing device according to Configuration Example 4, further including a detection unit configured to detect a movement of the subject, and a storage unit configured to store information about sleep of the subject, in which when the exercise intensity is equal to or greater than the second threshold value, the determination unit determines that the state of the subject is a second exercise state, the generation unit generates advice on an activity including a next day's exercise based on the first exercise state and the second exercise state, the acquisition unit acquires a wake-up time of the subject and a sleep time of the subject based on the movement and the biometric information of the subject, the determination unit determines whether a sleep time is sufficient for the exercise intensity for the day including the exercise intensity in the first exercise state and the second exercise state, and when it is determined that the sleep time is insufficient, the output unit outputs the advice on the activity including the next day's exercise at a fourth timing later than a third timing when the sleep time is determined to be sufficient.

### Configuration Example 9

The information processing device according to Configuration Example 4, in which when the exercise intensity is equal to or greater than the second threshold value, the determination unit determines that the state of the subject is a second exercise state, the generation unit generates advice to promote different exercises based on the second exercise state of the subject, and when the exercise intensity of the second exercise state satisfies a predetermined strength, the output unit outputs advice to promote different exercises.

### Configuration Example 10

The information processing device according to Configuration Example 9, in which the generation unit generates advice on review of the exercise based on the first exercise state and the second exercise state, and when it is determined that the exercise intensity is changed from a value equal to or greater than the second threshold value to a value smaller than the first threshold value, the output unit outputs the advice on review of the exercise after an elapse of a predetermined second time.

### Configuration Example 11

The information processing device according to Configuration Example 10, in which the predetermined second time of a case where the exercise intensity that is equal to or greater than the second threshold value and equal to or greater than a third threshold value is acquired by the acquisition unit is longer than the predetermined second time of a case where the exercise intensity that is equal to or greater than the second threshold value and equal to or greater than the third threshold value is not acquired by the acquisition unit.

### Configuration Example 12

The information processing device according to Configuration Example 1 or Configuration Example 2, in which the acquisition unit acquires an exercise amount based on the biometric information, when the exercise amount is a first exercise amount, the determination unit determines that the state of the subject is a first exercise state, the generation unit generates advice on an exercise based on the first exercise state, and the output unit outputs the advice on the exercise after an elapse of a predetermined third time after it is determined that the state is the first exercise state.

### Configuration Example 13

The information processing device according to Configuration Example 12, in which when the exercise amount is equal to or greater than a second exercise amount greater than the first exercise amount, the determination unit determines that the exercise state of the subject is a second exercise state, the generation unit generates advice on an activity including an exercise based on the second exercise state of the subject, and the output unit outputs the advice on the activity at a sixth timing earlier than a fifth timing in accordance with the first exercise state and the second exercise state.

### Configuration Example 14

The information processing device according to Configuration Example 13, in which the advice on the activity includes advice to the subject to promote sleep.

### Configuration Example 15

The information processing device according to Configuration Example 14, further including a detection unit configured to detect a movement of the subject, and a storage unit configured to store information about sleep of the subject, in which the acquisition unit acquires a bedtime or a wake-up time of the subject based on the movement and the biometric information of the subject, the storage unit stores the bedtime or the wake-up time of the subject up to the previous day, the sixth timing is a timing corresponding to the bedtime or the wake-up time of the subject up to the previous day.

### Configuration Example 16

The information processing device according to Configuration Example 12, further including a detection unit configured to detect a movement of the subject, and a storage unit configured to store information about sleep of the subject, in which when the exercise amount is equal to or greater than a second exercise amount greater than the first exercise amount, the determination unit determines that the exercise state of the subject is a second exercise state, the generation unit generates advice on an activity including a next day's exercise based on the first exercise state and the second exercise state, the acquisition unit acquires a wake-up time of the subject and a sleep time of the subject based on the movement and the biometric information of the subject, the determination unit determines whether the sleep time is sufficient for the day's exercise amount including the exercise amount in the first exercise state and the second exercise state, and when it is determined that the sleep time is insufficient, the output unit outputs the advice on the activity including the next day's exercise at an eighth timing later than a seventh timing based on the wake-up time of the subject up to the previous day.

### Configuration Example 17

The information processing device according to Configuration Example 12, in which when the exercise amount is equal to or greater than a second exercise amount greater than the first exercise amount, the determination unit determines that the exercise state of the subject is a second exercise state, the generation unit generates advice to promote different exercises based on the second exercise state of the subject, and when the exercise amount of the second exercise state satisfies a predetermined exercise amount, the output unit outputs the advice to promote different exercises.

### Configuration Example 18

The information processing device according to Configuration Example 17, in which the generation unit generates advice on review of the exercise based on the first exercise state and the second exercise state, and when it is determined that the exercise amount is changed from the exercise amount of the second exercise state to an amount equal to or smaller than the exercise amount in the first exercise state, the output unit outputs the advice on review of the exercise after an elapse of a predetermined fourth time.

### Configuration Example 19

The information processing device according to Configuration Example 18, in which the predetermined fourth time corresponding to a first total exercise amount in the first exercise state and the second exercise state is longer than the predetermined fourth time corresponding to a second total exercise amount smaller than the first total exercise amount.

### Configuration Example 20

The information processing device according to Configuration Example 4, in which the biometric information includes information about pulsation, the acquisition unit acquires data related to heart rate variability from the biometric information, and when there is a predetermined change in the data related to heart rate variability, the output unit outputs advice on an activity.

For example, it is possible to provide a method for performing an information process similar to the information process performed by the information processing device.

### Configuration Example 21

An information processing method including acquiring, by an acquisition unit, biometric information of a subject, determining, by a determination unit, a state of the subject from the biometric information, generating, by a generation unit, advice corresponding to the state of the subject, and outputting, by an output unit, the advice generated in such a manner that an output timing of advice is changed in accordance with the biometric information.

For example, it is possible to provide a recording medium storing a program with a function of performing an information process similar to the information process performed by the information processing device. This program is a computer program.

### Configuration Example 22

A recording medium configured to record a program, in which the program causes a computer to execute an acquisition function of acquiring biometric information of a subject, a determination function of determining a state of the subject from the biometric information, a generation function of generating advice corresponding to the state of the subject, and an output function of outputting the advice generated in such a manner that an output timing of advice is changed in accordance with the biometric information.

What is claimed is:

1. An information processing device comprising:
an acquisition unit that acquires biometric information of a subject;
a determination unit that determines a state of the subject from the biometric information;
a generation unit that generates advice corresponding to the state of the subject; and
an output unit that outputs the advice generated, in which the output unit changes an output timing of the advice in accordance with the biometric information.

2. The information processing device according to claim 1, wherein the biometric information includes at least any of pulsation, SpO2, and body temperature.

3. The information processing device according to claim 2, wherein
the acquisition unit acquires an exercise intensity based on the biometric information,
when the exercise intensity is equal to or greater than a first threshold value and is smaller than a second threshold value, the determination unit determines that the state of the subject is a first exercise state,
the generation unit generates advice on an exercise based on the first exercise state, and
the output unit outputs the advice on the exercise after an elapse of a predetermined first time after it is determined that the state is the first exercise state.

4. The information processing device according to claim 3, wherein the predetermined first time of a case where a maximum value of the exercise intensity is a first exercise intensity close to the first threshold value is shorter than the predetermined first time of a case where the maximum value of the exercise intensity is a second exercise intensity close to the second threshold value.

5. The information processing device according to claim 4, wherein
when the exercise intensity is equal to or greater than the second threshold value, the determination unit determines that the state of the subject is a second exercise state,
the generation unit generates advice on an activity including an exercise based on the second exercise state of the subject, and
the output unit outputs the advice on the activity at a second timing earlier or later than the first timing in accordance with the first exercise state and the second exercise state.

6. The information processing device according to claim 5, wherein the advice on the activity includes advice to the subject to promote sleep.

7. The information processing device according to claim 6, further comprising:
a detection unit that detects a movement of the subject; and
a storage unit that stores information about sleep of the subject, wherein
the acquisition unit acquires a bedtime or a wake-up time of the subject based on the movement and the biometric information of the subject,
the storage unit stores the bedtime or the wake-up time of the subject up to the previous day, and
the second timing is a timing corresponding to the bedtime or the wake-up time of the subject up to the previous day.

8. The information processing device according to claim 4, further comprising:
a detection unit that detects a movement of the subject; and
a storage unit that store information about sleep of the subject, wherein
when the exercise intensity is equal to or greater than the second threshold value, the determination unit determines that the state of the subject is a second exercise state,
the generation unit generates advice on an activity including a next day's exercise based on the first exercise state and the second exercise state,
the acquisition unit acquires a wake-up time of the subject and a sleep time of the subject based on the movement and the biometric information of the subject,
the determination unit determines whether the sleep time is sufficient for the exercise intensity for the day including the exercise intensity in the first exercise state and the second exercise state, and
when it is determined that the sleep time is insufficient, the output unit outputs the advice on the activity including the next day's exercise at a fourth timing later than a third timing when the sleep time is determined to be sufficient.

9. The information processing device according to claim 4, wherein
when the exercise intensity is equal to or greater than the second threshold value, the determination unit determines that the state of the subject is a second exercise state,
the generation unit generates advice to promote different exercises based on the second exercise state of the subject, and
when the exercise intensity of the second exercise state satisfies a predetermined strength, the output unit outputs advice to promote different exercises.

10. The information processing device according to claim 9, wherein
the generation unit generates advice on review of the exercise based on the first exercise state and the second exercise state, and
when it is determined that the exercise intensity is changed from a value equal to or greater than the second threshold value to a value smaller than the first threshold value, the output unit outputs the advice on review of the exercise after an elapse of a predetermined second time.

11. The information processing device according to claim 10, wherein the predetermined second time of a case where the exercise intensity that is equal to or greater than the second threshold value and equal to or greater than a third threshold value is acquired by the acquisition unit is longer than the predetermined second time of a case where the exercise intensity that is equal to or greater than the second threshold value and equal to or greater than the third threshold value is not acquired by the acquisition unit.

12. The information processing device according to claim 2, wherein
the acquisition unit acquires an exercise amount based on the biometric information,
when the exercise amount is a first exercise amount, the determination unit determines that the state of the subject is a first exercise state,
the generation unit generates advice on an exercise based on the first exercise state, and
the output unit outputs the advice on the exercise after an elapse of a predetermined third time after it is determined that the state is the first exercise state.

13. The information processing device according to claim 12, wherein
when the exercise amount is equal to or greater than a second exercise amount greater than the first exercise amount, the determination unit determines that the exercise state of the subject is a second exercise state,
the generation unit generates advice on an activity including an exercise based on the second exercise state of the subject, and
the output unit outputs the advice on the activity at a sixth timing earlier than a fifth timing in accordance with the first exercise state and the second exercise state.

14. The information processing device according to claim 13, wherein the advice on the activity includes advice to the subject to promote sleep.

15. The information processing device according to claim 14, further comprising:
a detection unit that detects a movement of the subject; and
a storage unit that stores information about sleep of the subject, wherein
the acquisition unit acquires a bedtime or a wake-up time of the subject based on the movement and the biometric information of the subject,
the storage unit stores the bedtime or the wake-up time of the subject up to the previous day,
the sixth timing is a timing corresponding to the bedtime or the wake-up time of the subject up to the previous day.

16. The information processing device according to claim 12, further comprising:
a detection unit that detects a movement of the subject; and
a storage unit that stores information about sleep of the subject, wherein
when the exercise amount is equal to or greater than a second exercise amount greater than the first exercise amount, the determination unit determines that the exercise state of the subject is a second exercise state,
the generation unit generates advice on an activity including a next day's exercise based on the first exercise state and the second exercise state,
the acquisition unit acquires a wake-up time of the subject and a sleep time of the subject based on the movement and the biometric information of the subject,
the determination unit determines whether the sleep time is sufficient for the day's exercise amount including the exercise amount in the first exercise state and the second exercise state, and
when it is determined that the sleep time is insufficient, the output unit outputs the advice on the activity including the next day's exercise at an eighth timing later than a seventh timing based on the wake-up time of the subject up to the previous day.

17. The information processing device according to claim 12, wherein
when the exercise amount is equal to or greater than a second exercise amount greater than the first exercise amount, the determination unit determines that the exercise state of the subject is a second exercise state,
the generation unit generates advice to promote different exercises based on the second exercise state of the subject, and
when the exercise amount of the second exercise state satisfies a predetermined exercise amount, the output unit outputs the advice to promote different exercises.

18. The information processing device according to claim 17, wherein
the generation unit generates advice on review of the exercise based on the first exercise state and the second exercise state, and
when it is determined that the exercise amount is changed from the exercise amount of the second exercise state to an amount equal to or smaller than the exercise amount in the first exercise state, the output unit outputs the advice on review of the exercise after an elapse of a predetermined fourth time.

19. The information processing device according to claim 18, wherein the predetermined fourth time corresponding to a first total exercise amount in the first exercise state and the second exercise state is longer than the predetermined fourth time corresponding to a second total exercise amount smaller than the first total exercise amount.

20. An information processing method comprising:
acquiring, by an acquisition unit, biometric information of a subject;
determining, by a determination unit, a state of the subject from the biometric information;
generating, by a generation unit, advice corresponding to the state of the subject; and
outputting, by an output unit, the advice generated in such a manner that an output timing of advice is changed in accordance with the biometric information.
